(19) United States
(12) Patent Application Publication
(10) Pub. No.: US 2022/0093121 A1
(43) Pub. Date: Mar. 24, 2022

(54) Detecting Depression Using Machine Learning Models on Human Speech Samples

(71) Applicants: Sruthi Kotlo, Naperville, IL (US); Divya Singh Lidder, Naperville, IL (US); Anjana Rao Ramachandran, Lisle, IL (US)

(72) Inventors: Sruthi Kotlo, Naperville, IL (US); Divya Singh Lidder, Naperville, IL (US); Anjana Rao Ramachandran, Lisle, IL (US)

(21) Appl. No.: 17/469,087

(22) Filed: Sep. 8, 2021

Related U.S. Application Data
(60) Provisional application No. 63/082,320, filed on Sep. 23, 2020.

(51) Int. Cl. G10L 25/66; G10L 25/30

(52) U.S. Cl. CPC G10L 25/66; G10L 25/30

(57) ABSTRACT

Methods and systems for utilizing machine learning to detect the presence of psychological conditions are provided. An example method may involve receiving, at a computing device, an input audio signal and determining, from the input audio signal, (i) acoustic features of the input audio signal, and (ii) a visual representation of the input audio signal. The example method may also involve providing the acoustic features to a first trained neural network; receiving, from the first trained neural network, a first prediction of a psychological condition associated with the input audio signal; providing the visual representation to a second trained neural network; and receiving, from the second trained neural network, a second prediction of the psychological condition associated with the input audio signal. The example method may further involve determining a composite prediction comprising a weighted combination of the first and second prediction and providing the composite prediction.

### CROSS-REFERENCE TO RELATED APPLICATIONS

The present application claims priority to U.S. Provisional Patent Application No. 63/082,320, filed on Sep. 23, 2020, the contents of which are entirely incorporated by reference herein.

### BACKGROUND

Clinical depression is a major issue that is frequently glossed over. Indeed, adolescents and adults alike who in fact possess depressive disorders regularly go untreated or undiagnosed by medical professionals. Often, this is because such individuals underrepresent their depressive symptoms to the medical professionals to avoid perceived medical costs and social stigma around mental illnesses. However, in the long run, these undiagnosed depressive disorders can cause severe health risks and loss in quality of life.

### SUMMARY

The embodiments herein present a system that applies machine learning techniques to an individual's voice sample to detect the presence of psychological conditions, such as clinical depression, associated with that individual. Given the social stigma around reporting mental illnesses, the disclosed approach can advantageously increase the detection of mental illnesses in a community, as the techniques herein enable individuals to self-diagnose psychological conditions without requiring high-stress interaction with a medical professional. Further, the embodiments herein can advantageously eliminate the subjectivity of individual medical professionals in assessing the presence of psychological conditions by replacing such subjective diagnosis with a more objective evaluation. Other advantages are also possible.

Accordingly, a first example embodiment may involve receiving, at a computing device, an input audio signal. The first example embodiment may also involve determining, by the computing device and from the input audio signal, (i) one or more acoustic features of the input audio signal, and (ii) a visual representation of the input audio signal. The first example embodiment may further involve providing, by the computing device, the one or more acoustic features to a first trained neural network, wherein the first trained neural network was trained, with a first training data set comprising audio signals and psychological conditions respectively associated with the audio signals, to form predictions of the psychological conditions from the audio signals. The first example embodiment may also involve receiving, from the first trained neural network, a first prediction of a psychological condition associated with the input audio signal. The first example embodiment may additionally involve providing, by the computing device, the visual representation to a second trained neural network, wherein the second trained neural network was trained, with a second training data set comprising visual images and psychological conditions respectively associated with the visual images, to form predictions of the psychological conditions from the visual images. The first example embodiment may further involve receiving, from the second trained neural network, a second prediction of the psychological condition associated with the input audio signal. The first example embodiment may additionally involve determining, at the computing device, a composite prediction comprising a weighted combination of the first and second prediction. The first example embodiment may also involve providing, by the computing device, the composite prediction.

A second example embodiment may involve obtaining, by a computing device, a training data set comprising audio signals and psychological conditions respectively associated with the audio signals. The second example embodiment may further involve determining, by the computing device and from the training data set, (i) one or more acoustic features of the audio signals, and (ii) visual representations of the audio signals. The second example embodiment may also involve, using the one or more acoustic features, training, by the computing device, a first neural network to form predictions of the psychological conditions associated with the audio signals. The second example embodiment may yet further involve, using the visual representations, training, by the computing device, a second neural network to form predictions of the psychological conditions associated with the audio signals. The second example embodiment may additionally involve, based at least in part on the training of the first and second neural networks, determining, at the computing device, weightings for combining outputs of the first and second neural networks. The second example embodiment may also involve providing, by the computing device, the first neural network, the second neural network, and the weightings to second computing device, wherein the second computing device is configured to (i) apply the first neural network on an input audio signal to generate a first prediction, (ii) apply the second neural network on the input audio signal to generate a second prediction, and (iii) apply the weightings on the first and second predictions to produce a weighted combination of the first and second predictions.

In a third example embodiment, an article of manufacture may include a non-transitory computer-readable medium, having stored thereon program instructions that, upon execution by a computing system, cause the computing system to perform operations in accordance with the first and/or second example embodiment.

In a fourth example embodiment, a computing device may include at least one processor, as well as memory and program instructions. The program instructions may be stored in the memory, and upon execution by the at least one processor, cause the computing device to perform operations in accordance with the first and/or second example embodiment.

In a fifth example embodiment, a system may include various means for carrying out each of the operations of the first and/or second example embodiment.

These, as well as other embodiments, aspects, advantages, and alternatives, will become apparent to those of ordinary skill in the art by reading the following detailed description, with reference where appropriate to the accompanying drawings. Further, this summary and other descriptions and figures provided herein are intended to illustrate embodiments by way of example only and, as such, that numerous variations are possible. For instance, structural elements and process steps can be rearranged, combined, distributed, eliminated, or otherwise changed, while remaining within the scope of the embodiments as claimed.

### BRIEF DESCRIPTION OF THE DRAWINGS

FIG. 1 depicts a computing device, in accordance with example embodiments.

FIG. 2 depicts a cloud-based computing infrastructure, in accordance with example embodiments.

FIG. 3 depicts a neural network, in accordance with example embodiments.

FIG. 4 depicts a supervised machine learning pipeline, in accordance with example embodiments.

FIG. 5 depicts a process for predicting the presence of a psychological conditions, in accordance with example embodiments.

FIG. 6A depicts elements of an acoustic processor, in accordance with example embodiments.

FIG. 6B depicts elements of a visual processor, in accordance with example embodiments.

FIG. 7 depicts a flow chart, in accordance with example embodiments.

FIG. 8 depicts a flow chart, in accordance with example embodiments.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features unless stated as such. Thus, other embodiments can be utilized and other changes can be made without departing from the scope of the subject matter presented herein.

Accordingly, the example embodiments described herein are not meant to be limiting. It will be readily understood that the aspects of the present disclosure, as generally described herein, and illustrated in the figures, can be arranged, substituted, combined, separated, and designed in a wide variety of different configurations. For example, the separation of features into “client” and “server” components may occur in a number of ways.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

### I. Example Computing Devices and Cloud-Based Computing Environments

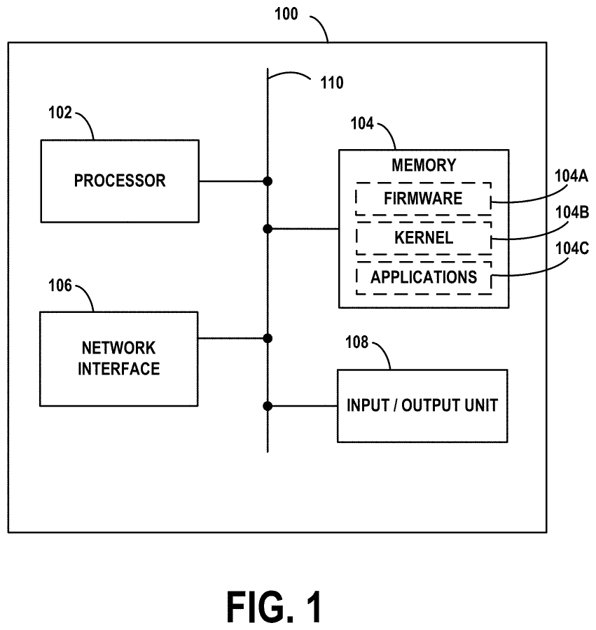

FIG. 1 is a simplified block diagram exemplifying a computing device 100, illustrating some of the components that could be included in a computing device arranged to operate in accordance with the embodiments herein. Computing device 100 could be a client device (e.g., a device actively operated by a user), a server device (e.g., a device that provides computational services to client devices), or some other type of computational platform. Some server devices may operate as client devices from time to time in order to perform particular operations, and some client devices may incorporate server features.

In this example, computing device 100 includes processor 102, memory 104, network interface 106, and input/output unit 108, all of which may be coupled by system bus 110 or a similar mechanism. In some embodiments, computing device 100 may include other components and/or peripheral devices (e.g., detachable storage, printers, and so on).

Processor 102 may be one or more of any type of computer processing element, such as a central processing unit (CPU), a co-processor (e.g., a mathematics, graphics, or encryption co-processor), a digital signal processor (DSP), a network processor, and/or a form of integrated circuit or controller that performs processor operations. In some cases, processor 102 may be one or more single-core processors. In other cases, processor 102 may be one or more multi-core processors with multiple independent processing units. Processor 102 may also include register memory for temporarily storing instructions being executed and related data, as well as cache memory for temporarily storing recently used instructions and data.

Memory 104 may be any form of computer-usable memory, including but not limited to random access memory (RAM), read-only memory (ROM), and non-volatile memory (e.g., flash memory, hard disk drives, solid state drives, compact discs (CDs), digital video discs (DVDs), and/or tape storage). Thus, memory 104 represents both main memory units, as well as long-term storage. Other types of memory may include biological memory.

Memory 104 may store program instructions and/or data on which program instructions may operate. By way of example, memory 104 may store these program instructions on a non-transitory, computer-readable medium, such that the instructions are executable by processor 102 to carry out any of the methods, processes, or operations disclosed in this specification or the accompanying drawings.

As shown in FIG. 1, memory 104 may include firmware 104A, kernel 104B, and/or applications 104C. Firmware 104A may be program code used to boot or otherwise initiate some or all of computing device 100. Kernel 104B may be an operating system, including modules for memory management, scheduling, and management of processes, input/output, and communication. Kernel 104B may also include device drivers that allow the operating system to communicate with the hardware modules (e.g., memory units, networking interfaces, ports, and buses) of computing device 100. Applications 104C may be one or more user-space software programs, such as web browsers or email clients, as well as any software libraries (e.g., scheduling algorithms and/or random number generators) used by these programs. Memory 104 may also store data used by these and other programs and applications.

Network interface 106 may take the form of one or more wireline interfaces, such as Ethernet (e.g., Fast Ethernet, Gigabit Ethernet, and so on). Network interface 106 may also support communication over one or more non-Ethernet media, such as coaxial cables or power lines, or over wide-area media. Network interface 106 may additionally take the form of one or more wireless interfaces, such as IEEE 802.11 (Wifi), BLUETOOTH®, global positioning system (GPS), or a wide-area wireless interface (e.g., MIMO-based 5G). However, other forms of physical layer interfaces and other types of standard or proprietary communication protocols may be used over network interface 106. Furthermore, network interface 106 may comprise multiple physical interfaces.

Input/output unit 108 may facilitate user and peripheral device interaction with computing device 100. Input/output unit 108 may include one or more types of input devices, such as a keyboard, a mouse, a touch screen, and so on. Similarly, input/output unit 108 may include one or more types of output devices, such as a screen, monitor, printer, and/or one or more light emitting diodes (LEDs). Additionally or alternatively, computing device 100 may communicate with other devices using a universal serial bus (USB) or high-definition multimedia interface (HDMI) port interface, for example.

One or more computing devices like computing device 100 may be deployed to support the embodiments herein. The exact physical location, connectivity, and configuration of these computing devices may be unknown and/or unimportant to client devices. Accordingly, the computing devices may be referred to as “cloud-based” devices that may be housed at various remote data center locations.

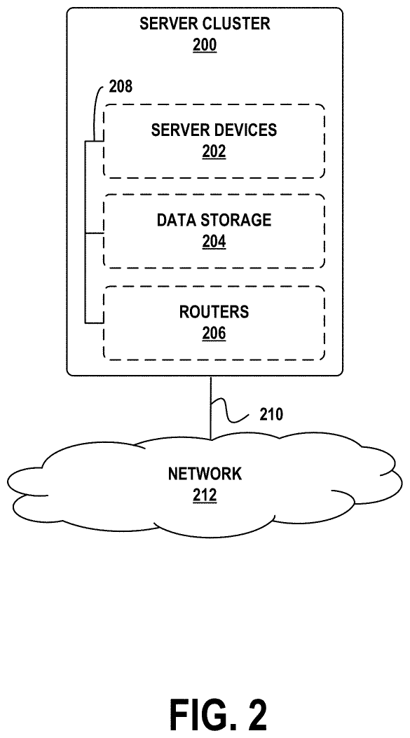

FIG. 2 depicts a cloud-based server cluster 200 in accordance with example embodiments. In FIG. 2, operations of a computing device (e.g., computing device 100) may be distributed between server devices 202, data storage 204, and routers 206, all of which may be connected by local cluster network 208. The number of server devices 202, data storages 204, and routers 206 in server cluster 200 may depend on the computing task(s) and/or applications assigned to server cluster 200.

For example, server devices 202 can be configured to perform various computing tasks of computing device 100. Thus, computing tasks can be distributed among one or more of server devices 202. To the extent that these computing tasks can be performed in parallel, such a distribution of tasks may reduce the total time to complete these tasks and return a result. For purposes of simplicity, both server cluster 200 and individual server devices 202 may be referred to as a “server device.” This nomenclature should be understood to imply that one or more distinct server devices, data storage devices, and cluster routers may be involved in server device operations.

Data storage 204 may be data storage arrays that include drive array controllers configured to manage read and write access to groups of hard disk drives and/or solid state drives. The drive array controllers, alone or in conjunction with server devices 202, may also be configured to manage backup or redundant copies of the data stored in data storage 204 to protect against drive failures or other types of failures that prevent one or more of server devices 202 from accessing units of data storage 204. Other types of memory aside from drives may be used.

Routers 206 may include networking equipment configured to provide internal and external communications for server cluster 200. For example, routers 206 may include one or more packet switching and/or routing devices (including switches and/or gateways) configured to provide (i) network communications between server devices 202 and data storage 204 via local cluster network 208, and/or (ii) network communications between server cluster 200 and other devices via communication link 210 to network 212.

Additionally, the configuration of routers 206 can be based at least in part on the data communication requirements of server devices 202 and data storage 204, the latency and throughput of the local cluster network 208, the latency, throughput, and cost of communication link 210, and/or other factors that may contribute to the cost, speed, fault-tolerance, resiliency, efficiency, and/or other design goals of the system architecture.

As a possible example, data storage 204 may include any form of database, such as a structured query language (SQL) database. Various types of data structures may store the information in such a database, including but not limited to tables, arrays, lists, trees, and tuples. Furthermore, any databases in data storage 204 may be monolithic or distributed across multiple physical devices.

Server devices 202 may be configured to transmit data to and receive data from data storage 204. This transmission and retrieval may take the form of SQL queries or other types of database queries, and the output of such queries, respectively. Additional text, images, video, and/or audio may be included as well. Furthermore, server devices 202 may organize the received data into web page or web application representations. Such a representation may take the form of a markup language, such as the hypertext markup language (HTML), the extensible markup language (XML), or some other standardized or proprietary format. Moreover, server devices 202 may have the capability of executing various types of computerized scripting languages, such as but not limited to Perl, Python, PHP Hypertext Preprocessor (PHP), Active Server Pages (ASP), JAVASCRIPT®, and so on. Computer program code written in these languages may facilitate the providing of web pages to client devices, as well as client device interaction with the web pages. Alternatively or additionally, JAVA® may be used to facilitate generation of web pages and/or to provide web application functionality.

Additionally, server devices 202 may be configured to carry out various types of machine learning training and/or execution tasks, such as those described below.

### II. Example Neural Networks

Generally speaking, an artificial neural network (ANN) (or “neural network” for short) is a computational model in which a number of simple units, working individually in parallel and often without central control, combine to solve complex problems. While this model may resemble an animal's brain in some respects, analogies between neural networks and brains are tenuous at best. Modern neural networks have a fixed structure, a mathematical learning process, are usually trained to solve one problem at a time, and are much smaller than their biological counterparts.

A neural network may be represented as a number of nodes that are arranged into a number of layers, with connections between the nodes of adjacent layers. The description herein generally applies to a feed-forward multilayer neural network, but similar structures and principles are used in convolutional neural networks, recurrent neural networks, graph neural networks, and recursive neural networks, for example.

Input values are introduced to the first layer of the neural network (the input layer), traverse some number of hidden layers, and then traverse an output layer that provides output values. A neural network may be a fully-connected network, in that nodes of each layer aside from the input layer receive input from all nodes in the previous layer. But partial connectivity between layers is also possible.

Connections between nodes represent paths through which intermediate values flow, and are each associated with a respective weight that is applied to the respective intermediate value. Each node performs an operation on its received values and their associated weights (e.g., values between 0 and 1, inclusive) to produce an output value. In some cases this operation may involve a dot-product sum of the products of each input value and associated weight. An activation function (e.g., a sigmoid, tanh or ReLU function) may be applied to the result of the dot-product sum to produce a scaled output value. Other operations are possible

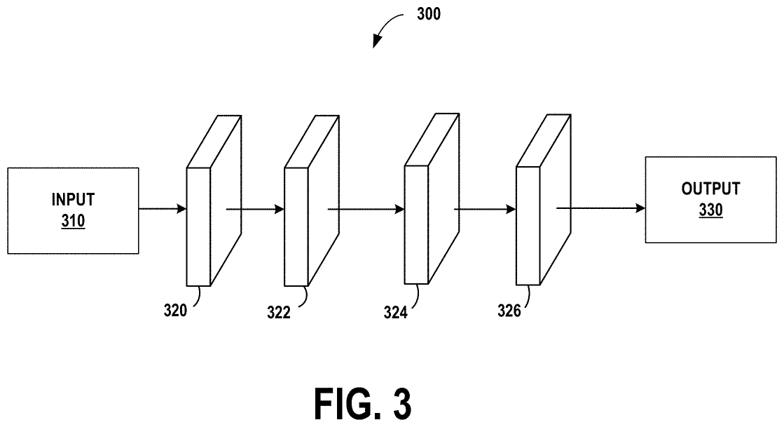

An example neural network is illustrated in FIG. 3. As shown, neural network 300 contains layer 320, layer 322, layer 324, and layer 326. During operations, neural network 300 may receive input 310, pass input 310 through layers 320, 322, 324, and 326, and produce output 330.

In some examples, layer 320 may contain 256 nodes and apply a ReLU activation function, layer 322 may contain 128 nodes and apply a ReLU activation function, layer 324 may contain 64 nodes and apply a ReLU activation function, and layer 326 may contain 2 nodes and apply a softmax activation function. In some instances, the layers of neural network 300 are fully connected (e.g., each node in layer 320 receives as input all features of input 310, each node in layer 322 receives as input all nodes of layer 320, etc . . . ).

While neural network 300 is shown to include four layers, in other embodiments, the number of layers in neural network 300 may vary. For instance, the number of layers in neural network 300 and the dimensions of those layers could depend on the size and number of layers in input 310 (e.g., the number of training samples passed to neural network 300). Other examples are also possible.

In some embodiments, neural network 300 may take the form of a convolutional neural network (CNN) that is configured to receive an input image and correspondingly generate a prediction based on that input image. To do this, neural network 300 may perform concatenation, convolution, activation, pooling, or inference tasks using a combination of concatenation layers, convolution layers, activation layers, pooling layers, and fully connected layers.

Generally speaking, a convolution layer includes one or more filters used to filter respective inputs. Each filter works over a subset of an input image or volume. For example, suppose an input to a convolutional layer was a 100×100-pixel image in CMYK format (Z=4). As such, the convolution layer would receive the 100×100×4 volume of pixels as an input volume and would convolve a 3×3×4 filter over the 100×100×4 volume. To do this, the convolution layer would slide the filter across the width and height of the input volume and compute dot products between the entries of the filter and the input at each position that the filter is on the input volume. As the convolution layer slides the filter, the filter generates a 2-dimensional feature map that gives the responses of that filter at every spatial position of the input volume. Multiple such filters could be used in a given convolution layer to produce multiple 2-dimensional feature maps. Further, multiple 2-dimensional feature maps could be combined to form a 3-dimensional feature map.

The output of the convolution layer (e.g., the feature maps mentioned above) can be provided as an input to an activation layer. The activation layer may be applied to determine which values of the feature map are to be provided to a subsequent layer. More generally, the activation function can determine whether the output of the convolution layer (e.g., the feature map) is to be provided to a subsequent layer. Activation layers could utilize sigmoid/logistic activation functions, hyperbolic tangent activation functions, or ReLU functions, among other possibilities.

### III. Example Machine Learning Pipelines

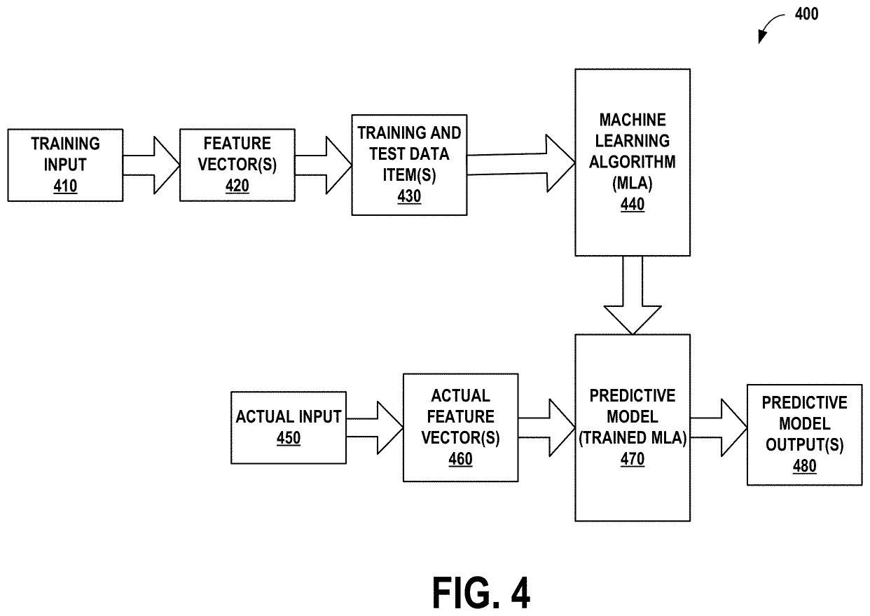

FIG. 4 is a diagram of a supervised learning pipeline 400, according to example embodiments. Supervised learning pipeline 400 includes training input 410, one or more feature vectors 420, one or more training and test data items 430, machine learning algorithm 440, actual input 450, one or more actual feature vectors 460, predictive model 470, and one or more predictive model outputs 480. Part or all of supervised learning pipeline 400 can be implemented by executing software for part or all of supervised learning pipeline 400 on one or more processing devices and/or by using other circuitry (e.g., computing device 100, server cluster 200).

In operation, supervised learning pipeline 400 can involve two phases: a training phase and a prediction phase. The training phase can involve machine learning algorithm 440 learning how to carry out one or more tasks. The prediction phase can involve predictive model 470, which can be a trained version of machine learning algorithm 440, making predictions to accomplish the one or more tasks. In some examples, machine learning algorithm 440 and/or predictive model 470 can include, but are not limited to, one or more: ANNs, deep neural networks (DNNs), CNNs, recurrent neural networks (RNNs), support vector machines (SVMs), Bayesian networks, genetic algorithms, linear classifiers, non-linear classifiers, algorithms based on kernel methods, logistic regression algorithms, linear discriminant analysis algorithms, and/or principal components analysis algorithms. In some examples, machine learning algorithm 440 and/or predictive model 470 can include all or some of the aspects of neural network 300, as described above with reference to FIG. 3.

During the training phase of supervised learning pipeline 400, training input 410 can be processed to determine one or more feature vectors 420. Then, feature vector(s) 420 (and/or training input 410) can be split into distinct training and test data items (refer to together as training and test data items 430). Of these, the training data items are used to train machine learning algorithm 440 and the test data items are used to determine how well machine learning algorithm 440 has been trained (e.g., the extent to which the use of machine learning algorithm 440 can be generalized beyond the training data items). As an example, 80% of training and test data items 430 may be used for training and 20% may be used for testing.

Thus, training and test data items 430 can be provided to machine learning algorithm 440 so that it can learn one or more tasks (e.g., classification of various inputs into a prediction of clinical depression. After performing the one or more tasks, machine learning algorithm 440 can generate one or more outputs based on feature vector(s) 420 and perhaps training input 410.

During training, training and test data item(s) 430 can be used to make an assessment of the output(s) of machine learning algorithm 440 for accuracy and machine learning algorithm 440 can be updated based on this assessment. For example, a loss function can be used to evaluate the error between the produced output values and training and test data item(s) 430. This loss function may be a sum of differences, mean squared error, or some other metric. In some cases, error values are determined for all of the sets of input values, and the error function involves calculating an aggregate (e.g., an average) of these values. Once the error is determined, the parameters of machine learning algorithm 440 (e.g., the weights on a neural network's connections) are updated in an attempt to reduce the error. In simple terms, this update process should reward “good” parameters and penalize “bad” parameters. Thus, the updating should distribute the “blame” for the error in a fashion that results in a lower error for future iterations of training.

Training of machine learning algorithm 440 can continue until machine learning algorithm 440 is considered to be trained to perform the one or more tasks. This occurs when the error is less than a threshold value or the change in the error is sufficiently small between consecutive iterations of training. Some training techniques, particularly for neural networks, may make use of some form of backpropagation. Backpropagation distributes the error for a neural network one layer at a time, from the output layer, through the hidden layers and to the input layer. Thus, the weights of the connections between the last hidden layer and the output layer are updated first, the weights of the connections between second-to-last hidden layer and last hidden layer are updated second, and so on. This updating can be based on a partial derivative of the activation function for each node and that node's connectivity to other nodes. Backpropagation completes when all weights have been updated.

In some cases, various hyperparameters can be used to adjust the training of machine learning algorithm 440. For example, constant biases can be applied to parameters of machine learning algorithm 440. Further, a multiplicative learning rate, or gain, could be applied when parameters are updated. Other possibilities exist.

While the discussion above assumes supervised training, training processes can also be unsupervised. For instance, given a corpus of data, machine learning algorithm 440 can learn mappings from this data to real-valued vectors in such a way that resulting vectors are similar for data with similar content. This can be achieved using, for example, auto-encoders that reconstruct the original vector from a smaller representation with reconstruction error as a cost function. This process creates meaningful representations that can be used for interpretability, for example.

Once trained, machine learning algorithm 440 can be considered to be a predictive model, such as predictive model 470.

During the prediction phase of supervised learning pipeline 400, actual input 450 can be processed to generate one or more actual feature vectors 460. Then, actual feature vectors 460 (and/or actual input 450) can be provided to predictive model 470. Predictive model 470 can generate one or more outputs, such as predictions, based on actual input 450/actual feature vectors 460. The output(s) of predictive model 470 can then be provided as predictive model output(s) 480. In some examples, predictive model 470 can receive a request to make one or more predictions, and reception of the request can trigger predictive model 470 to generate predictive model output(s) 480 based on actual input 450 and/or actual feature vector(s) 460. In some of these examples, the request can include and/or refer to actual input 450.

In some examples, machine learning algorithm 440 can be trained on one or more training computing devices and predictive model 470 can be executed on the same training computing device(s). In some examples, machine learning algorithm 440 can be trained on the training computing device(s). Then, after training, now-trained machine learning algorithm 440 can be communicated as predictive model 470 from the training computing device(s) to one or more other computing devices that can execute predictive model 470 to operate on actual input 450 to generate predictive model output(s) 480.

### IV. Example Systems for Predicting Psychological Conditions

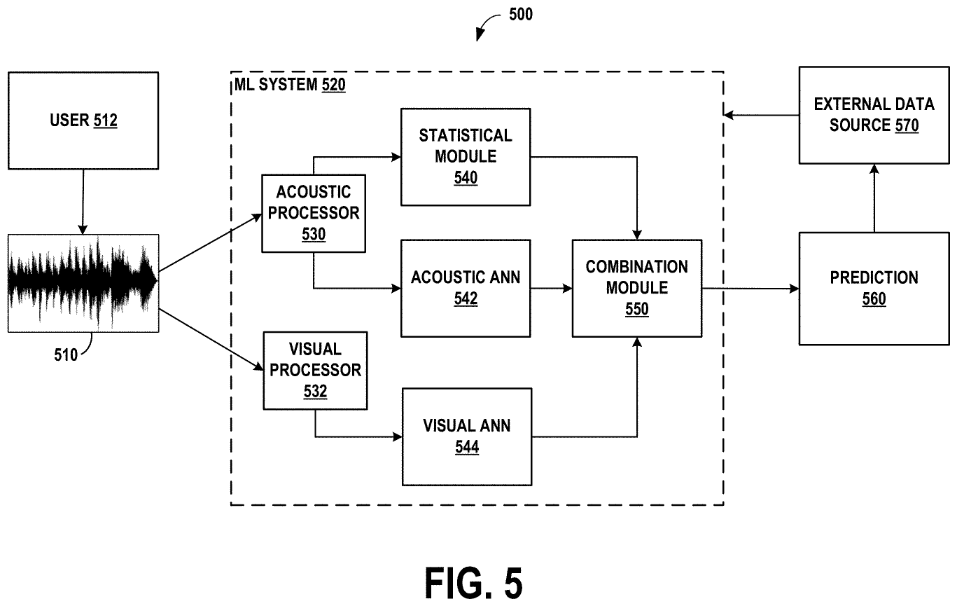

FIG. 5 is a diagram of process 500, according to example embodiments. In accordance with the disclosure, the embodiments of process 500 could be used to predict the presence of a psychological condition (e.g., the presence of clinical depression) for an input voice sample. As shown, process 500 includes input 510, machine learning (ML) system 520, and prediction 560. Part or all of process 500 can be implemented by executing software for part or all of process 500 on one or more processing devices and/or by using other circuitry (e.g., computing device 100, server cluster 200).

Process 500 may begin with input 510 being provided to ML system 520. In accordance with the disclosure, input 510 may be a voice sample from an individual seeking confirmation on whether they have an underlying psychological condition (e.g., whether they have clinical depression). In some examples, input 510 may take the form of an audio signal that is received by ML system 520 in waveform audio file format (WAV), though other forms of audio input may be possible.

In some embodiments, input 510 may be provided to ML system 520 by user 512. For instance, ML system 520 may prompt user 512 to enter an appropriate audio signal for input 510. This may be accomplished by way of a web page of a series of web pages hosted by the computing device and/or server cluster executing ML system 520 and provided by user 512 upon request. Using these web pages, user 512 can record and/or upload an audio file that is transmitted back to ML system 520. Alternatively, the user may provide this input by way of a mobile application that then forwards the information to ML system 520.

Because the pronunciation of certain words may better highlight the presence of psychological conditions, in some instances, the prompt offered by ML system 520 to user 512 may be a request for user 512 to vocalize a specific series of words. During operations, ML system 520 could adjust the series of prompted words to increase its predicative accuracy (i.e., maximize the predictive capability of ML system 520). For instance, if ML system 520 determines that the series of words currently offered to user 512 is yielding a predictive accuracy below a certain threshold (e.g., 60%, 70%, 80%, 90%), then ML system 520 could adjust the series of words accordingly until the predictive accuracy increases above that threshold.

In addition, ML system 520 may contain control mechanisms to ensure that user 512 is actually vocalizing the prompted series of words when they provide input 510 to ML system 520. For example, ML system 520 may contain an automatic speech recognition module that can recognize and translate the audio signal provided by user 512 into text. If the text does not exactly match the series of words, or if the text only matches a threshold low amount of the series of words (e.g., 60%, 70%, 80%, 90%), then ML system 520 may re-prompt user 512 to provide another audio signal.

Occasionally, the audio signal provided to ML system 520 contains underlying noise that might negatively affect the predictive capability of ML system 520. For instance, if user 512 records an audio file on their mobile device while walking on a busy city street, the background noise from the street may inadvertently be captured in that audio file. To reduce this noise, in some examples, ML system 520 may estimate, via spectral subtraction, Wiener filtering, or other methods, the amount of noise present in input 510. If the amount of noise is threshold high (e.g., the audio signal has a speech to noise ratio (SNR) of below 10 dB, below 0 dB, below −5 dB), then ML system 520 may re-prompt user 512 to provide another voice sample or may implement de-noising algorithms (e.g., DNN based algorithms, Wiener filtering based algorithms, spectral subtraction based algorithms) to reduce the amount of noise in the audio signal.

In addition to audio signals, in some embodiments, input 510 may include demographic data from user 512. For instance, input 510 may include the age, weight, ethnicity, education level, occupation, socio-economic status, or other types of information on user 512 that can assist ML system 520 in accurately diagnosing psychological conditions. The demographic data may be entered by user 512 via the same web page or series of web pages in which user 512 entered the audio signal. In such embodiments, ML system 520 may include an additional trained ANN (e.g., a predictive model 470) that was trained with a training data set (e.g., training input 410) comprising demographic data and ground truth values (e.g., training and test data items 430) comprising psychological conditions respectively associated with the demographic data, and combination module 550 may be further configured to combine the predictions made from the additional trained ANN to form prediction 560. Further details on combination module 550 are described below.

Upon receiving input 510, ML system 520 may pass instances of input 510 to acoustic processor 530 and visual processor 532.

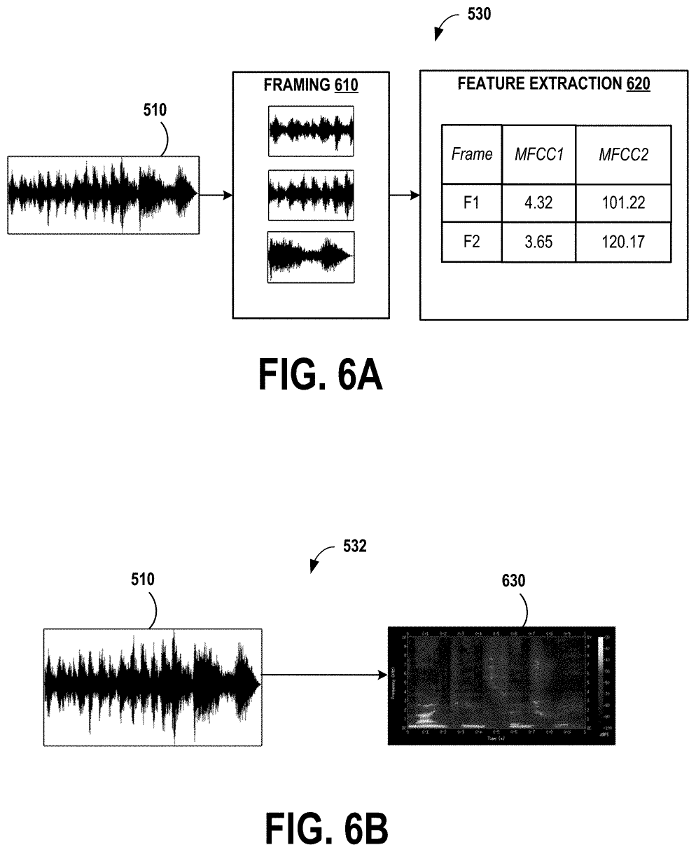

FIG. 6A illustrates elements of acoustic processor 530, in accordance with example embodiments. As shown, acoustic processor 530 contains two stages: framing stage 610 and feature extraction stage 620. During framing stage 610, acoustic processor 530 receives input 510 and divides input 510 into one or more frames (e.g., frames of 10 seconds each, 20 seconds each, 30 seconds each, etc.). For instance, if input 510 is an audio signal that is 60 seconds in length, framing stage 610 may divide input 510 into three audio signals of 20 seconds in length, with one frame capturing the first 20 seconds of input 510, another frame capturing the second 20 seconds of input 510, and another frame capturing the third 20 seconds of input 510.

The number of frames and/or the size of frames may vary. In some embodiments, the number of frames is set based on the length of input 510. For example, if input 510 is an audio signal with a length of N seconds, acoustic processor 530 may be configured to generate N/Z frames, where Z is predefined. In some other embodiments, the length of the frames is set based on the length of input 510. For example, if input 510 is an audio signal with a length of N seconds, acoustic processor 530 may be configured to generate frames of length N/X, where X is predefined. The number of frames and/or the size of the frames may be adjusted dynamically by ML system 520 depending on, for example, the predictive accuracy of ML system 520. For instance, if ML system 520 determines that the current number of frames and/or size of the frames by used by acoustic processor 530 is yielding a predictive accuracy below a certain threshold (e.g., 60%, 70%, 80%, 90%), then ML system 520 could adjust number of frames and/or size of the frames accordingly until the predictive accuracy increases above that threshold.

After framing stage 610, acoustic processor 530 continues its operations with feature extraction stage 620. Here, acoustic processor 530 could determine, for each frame of the frames determined in framing stage 610, one or more acoustic features that represent and/or describe either the audio signal captured by the frame or a transformed version of the audio signal captured by the frame. Example acoustic features include mel-frequency cepstral (MFC) coefficients, which represent the short-term power spectrum of the frame, based on a linear cosine transform of a log power spectrum on a nonlinear melscale frequency. As shown in FIG. 6A, for each frame, acoustic processor 530 may determine values for MFC coefficients, with the number of coefficients (e.g., the number of columns shown in feature extraction stage 620) ranging between 13-20. Other types of acoustic features could also be used, including linear predictive coding features, and discrete wavelet transform (DWT) features, among other possibilities.

FIG. 6B illustrates elements of visual processor 532, in accordance with example embodiments. As shown, visual processor 532 receives input 510 and then generates spectrogram 630, which is a visual representation of the spectrum of frequencies of input 510 over time. In particular, the y-axis of spectrogram 630 may represent frequency and the x-axis of spectrogram 630 may represent time. The color range of spectrogram 630 may be used to represent the amplitude of a particular frequency at a particular time.

Returning back to FIG. 5, after performing framing and feature extraction on input 510, acoustic processor 530 may provide the acoustic features it generated to statistical module 540 and acoustic ANN 542.

Statistical module 540 may be configured to select, from the acoustic features provided by acoustic process 530, a set of statistically relevant features. These statistical relevant features could be determined ahead of time (e.g., during a training phase) using various statistical processes, such as t-tests, multiple regression analysis, area under receiver operator curve (ROC), and/or other processes. With the set of selected statistically relevant features, statistical module 540 could then generate predictions of psychological condition using, for example, a statistical model, such as linear regression, non-linear regression, etc. As a particular example, statistical module 540 could determine, during a training phase, that MFC coefficients 3, 5, and 16 are the most statistically relevant for predicting the presence of psychological conditions associated with an audio signal (this has been the case in practical experiments with real world data). Then, during a prediction phase, statistical module 540 could select MFC coefficients 3, 5, and 16 from the acoustic features provided by acoustic process 530 and, using the values for MFC coefficients 3, 5, and 16, make a prediction of psychological conditions associated with input 510.

Acoustic ANN 542 could take the form of a trained ANN (e.g., a predictive model 470) that was trained with a training data set (e.g., training input 410) comprising acoustic features that represent and/or describe audio signals and ground truth values (e.g., training and test data items 430) comprising psychological conditions respectively associated with those audio signals. The training of acoustic ANN 542 could involve, for example, receiving a training set containing audio signals and psychological conditions associated with the audio signals, determining, for example using acoustic processor 530, one or more acoustic features of the audio signals, and then, using the one or more acoustic features, training acoustic ANN 542 to form predictions of psychological conditions associated with the audio signals. In some embodiments, the training data set used to train acoustic ANN 542 comprises training samples derived from the Distress Analysis Interview Corpus (DAIC), Wizard of-Oz (WOZ) database.

During operations, acoustic ANN 542 can receive the output from acoustic processor 530 (e.g., acoustic features) and correspondingly generate predictions of psychological conditions associated with input 510. In example embodiments, acoustic ANN 542 could take on some or all of the elements of neural network 300, as described above in reference to FIG. 3.

Visual ANN 544 could take the form of a trained ANN (e.g., a predictive model 470) that was trained with a training data set (e.g., training input 410) comprising visual representations of audio signals and ground truth values (e.g., training and test data items 430) comprising psychological conditions respectively associated with those visual representations. The training of visual ANN 544 could involve, for example, receiving a training set containing audio signals and psychological conditions associated with the audio signals, determining, for example using visual processor 532, visual representations of the audio signals, and then, using the visual representations, training visual ANN 544 to form predictions of psychological conditions associated with the audio signals. Similar to acoustic ANN 542, the training data set used to train visual ANN 544 could comprise training samples derived from the DAIC-WOZ database.

During operations, visual ANN 544 can receive the output from visual processor 532 (e.g., a spectrogram) and correspondingly generate a prediction of psychological conditions associated with input 510. In some embodiments, visual ANN 544 could take the form of a CNN and perform concatenation, convolution, activation, pooling, or inference tasks using a combination of concatenation layers, convolution layers, activation layers, pooling layers, and fully connected layers. The number of layers in visual ANN 544 and the dimensions of those layers may vary. In some embodiments, the number of layers in visual ANN 544 and the dimensions of those layers depend the length of input 510.

Combination module 550 may operate to combine the output from statistical module 540, acoustic ANN 542, and/or visual ANN 544. For example, after statistical module 540, acoustic ANN 542, and visual ANN 544 each respectively pass a predictive vector to combination module 550 (e.g., a vector containing the likelihood of user 512 having a specific psychological condition, such as clinical depression), combination module 550 may combine the predictive vectors in accordance with predefined weightings. These weightings may be predefined and programmed into combination module 550, or may be determined, at least in part, based on the training of acoustic ANN 542 and visual ANN 544. For instance, if during training, acoustic ANN 542 is shown to perform more accurately than visual ANN 544, then the weightings may be such that the output from acoustic ANN 542 is weighted more heavily than the output from visual ANN 544.

In some embodiments, combination module 550 may give a greatest weight to predictions from acoustic ANN 542, and the second greatest weight to predictions from visual ANN 544. As an example, if acoustic ANN 542 predicted that user 512 was 70% likely to have clinical depression, visual ANN 544 predicted that user 512 was 90% likely to have clinical depression, and statistical module 540 predicted that user 512 was 80% likely to have clinical depression, and the weightings were such that acoustic ANN was weighted by 0.5, visual ANN was weighted by 0.3, and statistical module 540 was weighted by 0.2, then the resulting combination would be: (0.7*0.5+0.9*0.3+0.8*0.2)/3=0.78. Such an example is not intended to be limiting, and other weightings are possible in the embodiments herein.

In some embodiments, rather than combining the output from statistical module 540, acoustic ANN 542, and/or visual ANN 544, combination module 550 could take the form of a neural network layer that is fully connected to the penultimate layer of acoustic ANN 542 and visual ANN 544. In such embodiments, a global softmax activation function could be applied by combination module 550 to produce prediction 560, rather than, for instance, separate softmax activation functions being applied by the final layers of acoustic ANN 542 and visual ANN 544. In such a way, combination module 550 could be thought of as the final output layer of both acoustic ANN 542 and visual ANN 544.

The results from combination module 550 may be output as prediction 560. In some embodiments, prediction 560 contains a single binary prediction (i.e., Yes or No) on whether user 512 (by way of input 510) has a specific psychological condition. In such embodiments, acoustic ANN 542 and visual ANN 544 may be trained as binary classifiers, for instance, with ground truth training data containing binary classifications of psychological conditions. In other examples, prediction 560 may contain confidence score (e.g., 60% confident, 70% confident) on whether user 512 (by way of input 510) has a specific psychological condition. For instance, combination module 550 could apply a softmax function with two output classes, one representing the presence of a specific psychological condition (e.g., clinical depression) and one representing the absence of that specific psychological condition. During operations, the softmax function could return posterior probabilities for each output classes (e.g., 30% that there is no psychological condition, 70% that there is a psychological condition), which could then be used by combination module 550 as confidence scores. In some embodiments, if the confidence score is below a certain threshold (e.g., below 60%, 70%, 80%), ML system 520 may re-prompt user 512 to enter a second voice sample. To ensure higher predictive accuracy for the second voice sample, in some embodiments, the ML system 520 may request that the second voice sample be longer in length than the initial voice sample provided by user 512. If the confidence score is above the threshold, prediction 560 may be provided back to user 512 by way of the aforementioned web page or series of web pages hosted by the computing device and/or server cluster executing ML system 520.

Additionally, and/or alternatively, prediction 560 may be provided to external data source 570, which may take the form of a remote computing device or server cluster communicatively connected to ML system 520. In some embodiments, external data source 570 may take the form of an electronic health record (EHR) system configured to receive and record medical information related to user 512. In such embodiments, external data source 570 may be able to confirm the accuracy of prediction 560 and then transmit that confirmation back to ML system 520. For example, the operator of external data source 570 (e.g., a healthcare provider) may diagnose user 512 with certain psychological conditions and then may confirm whether prediction 560 matches those diagnosed conditions. If a match is found, the operator of external data source 570 may send a confirmation to ML system 520 that prediction 560 was correct. ML system 520 could then create a supplementary training sample using that confirmation. That is, ML system 520 could generate a training sample comprising input 510 as the feature vector and the psychological condition confirmed by external data source 570 as the ground truth value. After generating a threshold amount such supplementary training samples (e.g., 100, 200, 500, 1000), ML system 520 could retrain acoustic ANN 542 and/or visual ANN 544 using at least some of the supplementary training samples.

Note that the operations of process 500 should not be limited to the specific implementation described with respect to FIG. 5. For instance, in some embodiments, rather than applying each of statistical module 540, acoustic ANN 542, and visual ANN 544 onto input 510 to determine prediction 560, process 500 may involve the use of only one (or two) of statistical module 540, acoustic ANN 542, and visual ANN 544. Further, in other embodiments, process 500 may involve other types of ANNs and/or statistical modules.

### V. Example Operations

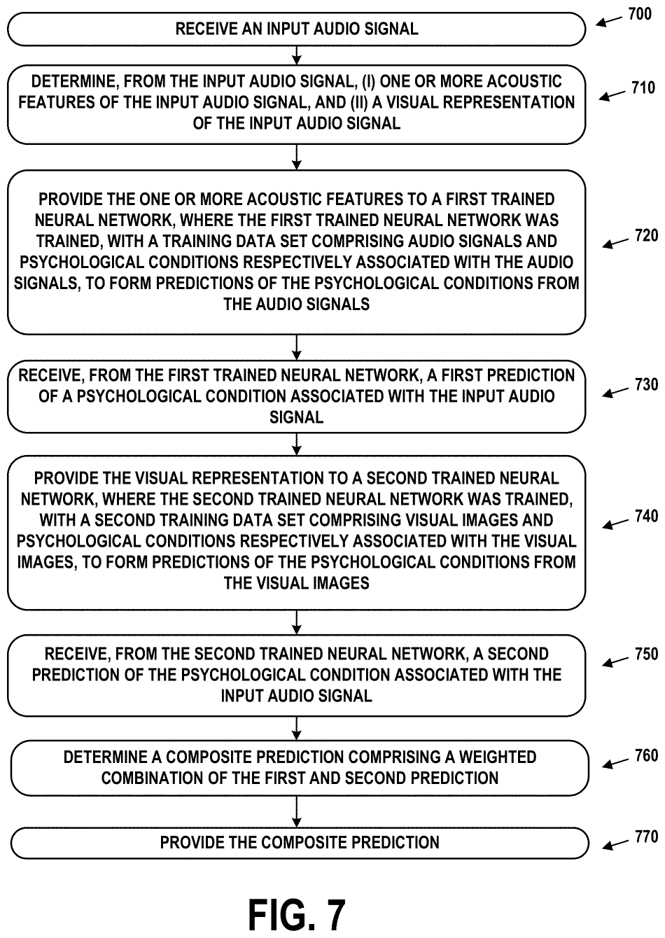

FIG. 7 is a flow chart illustrating an example embodiment. The process illustrated by FIG. 7 may be carried out by a computing device, such as computing device 100, and/or a cluster of computing devices, such as server cluster 200. However, the process can be carried out by other types of devices or device subsystems.

The embodiments of FIG. 7 may be simplified by the removal of any one or more of the features shown therein. Further, these embodiments may be combined with features, aspects, and/or implementations of any of the previous figures or otherwise described herein.

Block 700 involves receiving an input audio signal.

Block 710 involves determining from the input audio signal, (i) one or more acoustic features of the input audio signal, and (ii) a visual representation of the input audio signal.

Block 720 involves providing the one or more acoustic features to a first trained neural network, wherein the first trained neural network was trained with a first training data set comprising audio signals and psychological conditions respectively associated with the audio signals, to form predictions of the psychological conditions from the audio signals.

Block 730 involves receiving, from the first trained neural network, a first prediction of a psychological condition associated with the input audio signal.

Block 740 involves providing the visual representation to a second trained neural network, wherein the second trained neural network was trained with a second training data set comprising visual images and psychological conditions respectively associated with the visual images, to form predictions of the psychological conditions from the visual images.

Block 750 involves receiving, from the second trained neural network, a second prediction of the psychological condition associated with the input audio signal.

Block 760 involves determining a composite prediction comprising a weighted combination of the first and second prediction.

Block 770 involves providing the composite prediction.

In some embodiments, determining the one or more acoustic features includes segmenting the input audio signal into a number of frames and determining, respectively for each frame in the number of frames, at least one acoustic feature.

In some embodiments, the number of frames is set based on a length of the input audio signal.

Some embodiments may involve selecting, by way of a statistical module, a set of statistically relevant features from the one or more acoustic features and, using the set of statistically relevant features, determining a third prediction of the psychological condition associated with the input audio signal, wherein the weighted combination is of the first, second, and third prediction. In some embodiments, the first prediction is given greater weight in the weighted combination than the second and third predictions.

Some embodiments may involve transmitting, to a remote computing device, the composite prediction; receiving, from the remote computing device, information confirming that the composite prediction was accurate; and generating a supplementary training sample comprising the input audio signal and the psychological condition represented by the composite prediction.

Some embodiment may involve, after generating a threshold amount of supplementary training samples, retraining the first and second trained neural network with at least some of the supplementary training samples.

Some embodiments may involve receiving demographic data related to a source of the input audio signal; and using the demographic data, determining a third prediction of the psychological condition associated with the input audio signal, wherein the weighted combination is of the first, second, and third predictions.

In some embodiments, receiving the input audio signal includes receiving a first audio signal; determining whether the first audio signal contains threshold high noise levels; in response to determining that the first audio signal contains threshold high noise levels, requesting a second audio signal; and in response to determining that the second audio signal does not contain threshold high noise levels, using the second audio signal as the input audio signal.

In some embodiments, receiving the input audio signal includes transmitting a representation of a graphical interface to a remote computing device; and receiving, from the remote computing device and by way of the graphical interface, the input audio signal.

In some embodiments, the graphical interface includes a prompt requesting that the input audio signal contain vocalizations of a series of predefined words. Such embodiments may further include determining, using an automatic speech recognition module, that the input audio signal does not contain vocalizations of the series of predefined words; in response to determining that the input audio signal does not contain vocalizations of the series of predefined words, requesting a second audio signal; and after receiving the second audio signal, using the second audio signal as the input audio signal.

In some embodiments, the composite prediction comprises a confidence score, and providing the composite prediction includes determining that the confidence score is below a predefined threshold, and in response to the determining that the confidence score is below the predefined threshold, requesting a second input audio signal that is longer in length than the input audio signal.

Notably, in alternative embodiments, any of the first neural network, the second neural network or the statistical module could be used in isolation and without the others to form predictions.

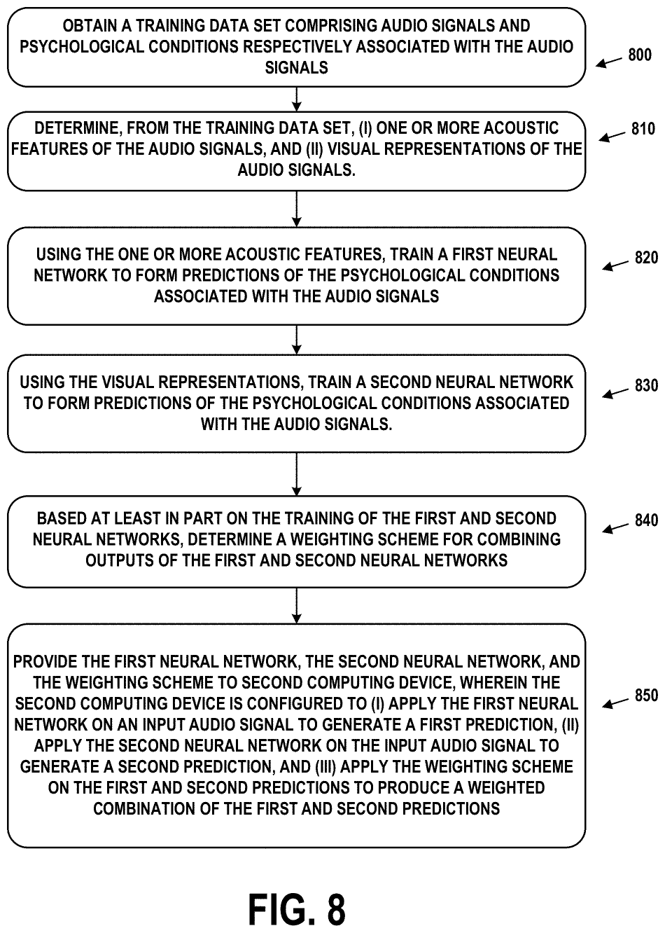

FIG. 8 is a flow chart illustrating an example embodiment. The process illustrated by FIG. 8 may be carried out by a computing device, such as computing device 100, and/or a cluster of computing devices, such as server cluster 200. However, the process can be carried out by other types of devices or device subsystems.

The embodiments of FIG. 8 may be simplified by the removal of any one or more of the features shown therein. Further, these embodiments may be combined with features, aspects, and/or implementations of any of the previous figures or otherwise described herein.

Block 800 involves obtaining a training data set comprising audio signals and psychological conditions respectively associated with the audio signals.

Block 810 involves determining, from the training data set, (i) one or more acoustic features of the audio signals, and (ii) visual representations of the audio signals.

Block 820 involves, using the one or more acoustic features, training a first neural network to form predictions of the psychological conditions associated with the audio signals.

Block 830 involves, using the visual representations, training a second neural network to form predictions of the psychological conditions associated with the audio signals.

Block 840 involves, based at least in part on the training of the first and second neural networks, determining weightings for combining outputs of the first and second neural networks.

Block 850 involves providing the first neural network, the second neural network, and the weightings to second computing device, wherein the second computing device is configured to (i) apply the first neural network on an input audio signal to generate a first prediction, (ii) apply the second neural network on the input audio signal to generate a second prediction, and (iii) apply the weightings on the first and second predictions to produce a weighted combination of the first and second predictions.

In some embodiments, a statistical module may also be determined from the training data set. In these embodiments, the one or more acoustic features and their associated psychological conditions may be used to identify statistically relevant features of the one or more acoustic features that are correlated with and/or predictive of the psychological conditions. The weighting scheme may combine output from the statistical module as well. The statistical module may also be provided to the second computing device, wherein the second computing device is also configured to apply the statistical module on the input audio signal to generate a third prediction that is provided to the weighting scheme.

The statistical module may determine that MFC coefficients 3, 5, and 16 are the most correlated with and/or predictive of the psychological conditions.

Notably, in alternative embodiments, any of the first neural network, the second neural network or the statistical module could be used in isolation and without the others to form predictions.

### VI. Conclusion

The present disclosure is not to be limited in terms of the particular embodiments described in this application, which are intended as illustrations of various aspects. Many modifications and variations can be made without departing from its scope, as will be apparent to those skilled in the art. Functionally equivalent methods and apparatuses within the scope of the disclosure, in addition to those described herein, will be apparent to those skilled in the art from the foregoing descriptions. Such modifications and variations are intended to fall within the scope of the appended claims.

The above detailed description describes various features and operations of the disclosed systems, devices, and methods with reference to the accompanying figures. The example embodiments described herein and in the figures are not meant to be limiting. Other embodiments can be utilized, and other changes can be made, without departing from the scope of the subject matter presented herein. It will be readily understood that the aspects of the present disclosure, as generally described herein, and illustrated in the figures, can be arranged, substituted, combined, separated, and designed in a wide variety of different configurations.

With respect to any or all of the message flow diagrams, scenarios, and flow charts in the figures and as discussed herein, each step, block, and/or communication can represent a processing of information and/or a transmission of information in accordance with example embodiments. Alternative embodiments are included within the scope of these example embodiments. In these alternative embodiments, for example, operations described as steps, blocks, transmissions, communications, requests, responses, and/or messages can be executed out of order from that shown or discussed, including substantially concurrently or in reverse order, depending on the functionality involved. Further, more or fewer blocks and/or operations can be used with any of the message flow diagrams, scenarios, and flow charts discussed herein, and these message flow diagrams, scenarios, and flow charts can be combined with one another, in part or in whole.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical operations or actions in the method or technique. The program code and/or related data can be stored on any type of computer readable medium such as a storage device including RAM, a disk drive, a solid state drive, or another storage medium.

The computer readable medium can also include non-transitory computer readable media such as computer readable media that store data for short periods of time like register memory and processor cache. The computer readable media can further include non-transitory computer readable media that store program code and/or data for longer periods of time. Thus, the computer readable media may include secondary or persistent long term storage, like ROM, optical or magnetic disks, solid state drives, or compact-disc read only memory (CD-ROM), for example. The computer readable media can also be any other volatile or non-volatile storage systems. A computer readable medium can be considered a computer readable storage medium, for example, or a tangible storage device.

Moreover, a step or block that represents one or more information transmissions can correspond to information transmissions between software and/or hardware modules in the same physical device. However, other information transmissions can be between software modules and/or hardware modules in different physical devices.

The particular arrangements shown in the figures should not be viewed as limiting. It should be understood that other embodiments can include more or less of each element shown in a given figure. Further, some of the illustrated elements can be combined or omitted. Yet further, an example embodiment can include elements that are not illustrated in the figures.

While various aspects and embodiments have been disclosed herein, other aspects and embodiments will be apparent to those skilled in the art. The various aspects and embodiments disclosed herein are for purpose of illustration and are not intended to be limiting, with the true scope being indicated by the following claims.

What is claimed is:

1. A computer-implemented method comprising:
receiving, at a computing device, an input audio signal;
determining, by the computing device and from the input audio signal, (i) one or more acoustic features of the input audio signal, and (ii) a visual representation of the input audio signal;
providing, by the computing device, the one or more acoustic features to a first trained neural network, wherein the first trained neural network was trained, with a first training data set comprising audio signals and psychological conditions respectively associated with the audio signals, to form predictions of the psychological conditions from the audio signals;
receiving, from the first trained neural network, a first prediction of a psychological condition associated with the input audio signal;
providing, by the computing device, the visual representation to a second trained neural network, wherein the second trained neural network was trained, with a second training data set comprising visual images and psychological conditions respectively associated with the visual images, to form predictions of the psychological conditions from the visual images;
receiving, from the second trained neural network, a second prediction of the psychological condition associated with the input audio signal;
determining, at the computing device, a composite prediction comprising a weighted combination of the first and second prediction; and
providing, by the computing device, the composite prediction.

2. The computer-implemented method of claim 1, wherein determining the one or more acoustic features comprises:
segmenting the input audio signal into a number of frames; and
determining, respectively for each frame in the number of frames, at least one acoustic feature.

3. The computer-implemented method of claim 2, wherein the number of frames is set based on a length of the input audio signal.

4. The computer-implemented method of claim 1, further comprising:
selecting, by way of a statistical module, a set of statistically relevant features from the one or more acoustic features; and
using the set of statistically relevant features, determining a third prediction of the psychological condition associated with the input audio signal, wherein the weighted combination is of the first, second, and third prediction.

5. The computer-implemented method of claim 4, wherein the first prediction is given greater weight in the weighted combination than the second and third predictions.

6. The computer-implemented method of claim 1, further comprising:
transmitting, from the computing device and to a remote computing device, the composite prediction;
receiving, at the computing device and from the remote computing device, information confirming that the composite prediction was accurate; and
generating, by the computing device, a supplementary training sample comprising the input audio signal and the psychological condition represented by the composite prediction.

7. The computer-implemented method of claim 6, further comprising:
after generating a threshold amount of supplementary training samples, retraining, by the computing device, the first and second trained neural network with at least some of the supplementary training samples.

8. The computer-implemented method of claim 1, further comprising:
receiving demographic data related to a source of the input audio signal; and
using the demographic data, determining a third prediction of the psychological condition associated with the input audio signal, wherein the weighted combination is of the first, second, and third predictions.

9. The computer-implemented method of claim 1, wherein receiving the input audio signal comprises:
receiving a first audio signal;
determining whether the first audio signal contains threshold high noise levels;
in response to determining that the first audio signal contains threshold high noise levels, requesting a second audio signal; and
in response to determining that the second audio signal does not contain threshold high noise levels, using the second audio signal as the input audio signal.

10. The computer-implemented method of claim 1, wherein receiving the input audio signal comprises:
transmitting, by the computing device, a representation of a graphical interface to a remote computing device; and
receiving, from the remote computing device and by way of the graphical interface, the input audio signal.

11. The computer-implemented method of claim 10, wherein the graphical interface includes a prompt requesting that the input audio signal contain vocalizations of a series of predefined words, and wherein the method further comprises:
determining, using an automatic speech recognition module, that the input audio signal does not contain vocalizations of the series of predefined words;
in response to determining that the input audio signal does not contain vocalizations of the series of predefined words, requesting a second audio signal; and
after receiving the second audio signal, using the second audio signal as the input audio signal.

12. The computer-implemented method of claim 1, wherein the composite prediction comprises a confidence score, and wherein providing the composite prediction comprises:
determining that the confidence score is below a predefined threshold, and in response to the determining that the confidence score is below the predefined threshold, requesting a second input audio signal that is longer in length than the input audio signal.

13. A computing device comprising:
one or more processors;
memory; and
program instructions, stored in the memory, that upon execution by the one or more processors cause the computing device to perform operations comprising:
receiving an input audio signal
determining, by the computing device and from the input audio signal, (i) one or more acoustic features of the input audio signal, and (ii) a visual representation of the input audio signal
providing the one or more acoustic features to a first trained neural network, wherein the first trained neural network was trained, with a first training data set comprising audio signals and psychological conditions respectively associated with the audio signals, to form predictions of the psychological conditions from the audio signals
receiving, from the first trained neural network, a first prediction of a psychological condition associated with the input audio signal
providing the visual representation to a second trained neural network, wherein the second trained neural network was trained, with a second training data set comprising visual images and psychological conditions respectively associated with the visual images, to form predictions of the psychological conditions from the visual images
receiving, from the second trained neural network, a second prediction of the psychological condition associated with the input audio signal;
determining a composite prediction comprising a weighted combination of the first and second prediction; and
providing the composite prediction.

14. The computing device of claim 13, wherein determining the one or more acoustic features comprises:
segmenting the input audio signal into a number of frames; and
determining, respectively for each frame in the number of frames, at least one acoustic feature.

15. The computing device of claim 13, wherein the operations further comprise:
selecting a set of statistically relevant features from the one or more acoustic features; and
using the set of statistically relevant features, determining a third prediction of the psychological condition associated with the input audio signal, wherein the weighted combination is of the first, second, and third prediction.

16. The computing device of claim 15, wherein the first prediction is given greater weight in the weighted combination than the second and third predictions.

17. The computing device of claim 13, wherein the operations further comprise:
transmitting, to a remote computing device, the composite prediction;
receiving, from the remote computing device, information confirming that the composite prediction was accurate; and
generating a supplementary training sample comprising the input audio signal and the psychological condition represented by the composite prediction.

18. The computing device of claim 17, wherein the operations further comprise:
after generating a threshold amount of supplementary training samples, retraining the first and second trained neural network with at least some of the supplementary training samples.

19. The computing device of claim 13, wherein receiving the input audio signal comprises:
transmitting a representation of a graphical interface to a remote computing device; and
receiving, from the remote computing device and by way of the graphical interface, the input audio signal.

20. A computer-implemented method comprising:
obtaining, by a computing device, a training data set comprising audio signals and psychological conditions respectively associated with the audio signals;
determining, by the computing device and from the training data set, (i) one or more acoustic features of the audio signals, and (ii) visual representations of the audio signals;
using the one or more acoustic features, training, by the computing device, a first neural network to form predictions of the psychological conditions associated with the audio signals;
using the visual representations, training, by the computing device, a second neural network to form predictions of the psychological conditions associated with the audio signals;
based at least in part on the training of the first and second neural networks, determining, at the computing device, weightings for combining outputs of the first and second neural networks; and
providing, by the computing device, the first neural network, the second neural network, and the weightings to a second computing device, wherein the second computing device is configured to (i) apply the first neural network on an input audio signal to generate a first prediction, (ii) apply the second neural network on the input audio signal to generate a second prediction, and (iii) apply the weightings on the first and second predictions to produce a weighted combination of the first and second predictions.
